(19) United States
(12) Patent Application Publication
(10) Pub. No.: US 2019/0022266 A1
(43) Pub. Date: Jan. 24, 2019

(54) BOTTLE ESSENTIAL OIL DIFFUSER WITH A BREATHING FILM INCORPORATED CAP

(71) Applicant: DONGGUAN YIH TEH ELECTRIC PRODUCTS CO., LTD., Dongguan City (CN)

(72) Inventor: Ming Jen HSIAO, Miaoli County (TW)

(21) Appl. No.: 15/871,315

(22) Filed: Jan. 15, 2018

Related U.S. Application Data
(63) Continuation-in-part of application No. 15/653,362, filed on Jul. 18, 2017.

(51) Int. Cl. A61L 9/03

(52) U.S. Cl. CPC A61L 9/03; A61L 2209/131

(57) ABSTRACT

A bottle essential oil diffuser includes a bottle having a bottle mouth, an essential oil substance contained in the bottle, a cap capped on the bottle mouth of the bottle and defining therein a top opening and a breathing film consisting of a fiber fixation layer and a microporous layer and bonded to the cap over the top opening for allowing generated aromatic molecular vapor to pass thereof and prohibiting the essential oil substance from flowing out of the top opening of the cap.

### CROSS-REFERENCES TO RELATED APPLICATION

The present invention is a continuation-in-part of patent application Ser. No. 15/653,362 filed on Jul. 18, 2017.

### BACKGROUND OF THE INVENTION

### 1. Field of the Invention

The present invention relates to scent releasing devices and more specifically, to a bottle essential oil diffuser with a breathing film incorporated cap, which prevents the essential oil from spilling out from the bottle during application.

### 2. Description of the Related Art

In application of the aroma-diffusing heating device designs of U.S. Pat. No. 20140014736A1, U.S. Pat. No. 8,668,885, U.S. Pat. No. 9,550,358, U.S. Pat. No. 9,498,553 and EP Pat. No. 2679249 that are invented by the present inventor, when the heat-transfer container is heated, the heat energy is transferred to the breathing film. Since the breathing film is a plastic film having porous therein, it can be deformed after a long use, causing wrinkles or even affecting the flowing of the generated aromatic molecular vapor therethrough toward the outside open air.

However, when using a conventional essential oil diffuser, the essential oil can spill out from the diffuser. Therefore, at present, the safety regulation provides that the essential oil can not spill out from the diffuser during application. If an essential oil diffuser falls down as it is heated by a heater to generate an aromatic vapor, the essential oil can spill out from the diffuser to contaminate the heater and the surroundings or to cause a dangerous condition.

### SUMMARY OF THE INVENTION

The present invention has been accomplished under the circumstances in view. It is therefore the main object of the present invention to provide a bottle essential oil diffuser with a breathing film incorporated cap that eliminates the drawbacks of the conventional aroma diffuser designs.

The invention has the advantages that the breathing film allows the aromatic molecular vapor generated by the essential oil substance to flow to the outside of the bottle essential oil diffuser and prohibits the essential oil substance from flowing out of the bottle essential oil diffuser.

In one embodiment, the bottle essential oil diffuser uses a heater to heat the essential oil substance. In the heating process, the breathing film is constantly kept in shape without wrinkles, allowing generated aromatic molecular vapor to smoothly flow therethrough to the outside open air. Even if the heater or the bottle essential oil diffuser falls down, the breathing film can effectively prohibit the fluid essential oil substance from flowing out of the top opening of the cap, avoiding contamination.

In one embodiment, the bottle essential oil diffuser comprised a bottle having a bottle mouth, an essential oil substance contained in the bottle, a cap capped on the bottle mouth of the bottle and defining therein a top opening, and a breathing film bonded to the cap over the top opening

Preferably, the breathing film comprises a fiber fixation layer and a microporous layer bonded together. The fiber fixation layer comprises a plurality of first pores. The microporous layer comprises a plurality of second pores. The microporous layer has a waterproof and moisture-permeable function. The fiber fixation layer is heat and deformation resistant. The breathing film allows the aromatic molecules of the essential oil substance to flow to the outside of the bottle essential oil diffuser and prohibits the essential oil substance from flowing out of the bottle essential oil diffuser during application.

In one embodiment of the present invention, the bottle essential oil diffuser further comprises a heater adapted for holding and heating the bottle, causing the essential oil substance to generate an aromatic vapor.

Preferably, the microporous layer is made from thermoplastic elastomer (TPE), and the fiber fixation layer is made from polyester. Preferably, the polyester is selected from the group consisting of Tetoron and polyethylene terephthalate (PET); said thermoplastic elastomer (TPE) is thermoplastic polyurethane (TPU). Preferably, the cap is made from a plastic material selected from the group consisting of polyester plastic, poly-cyclohexylenedimethulene terephthalate glyco (PCTG), polyethylene terephthalate (PET), poly-thiourethane (PTU) and polypropylene (PP). Further, the microporous layer of the breathing film is bonded to the top opening of the cap.

Preferably, the breathable aperture of the first pores is greater than the breathable aperture of the second pores, i.e., the breathable volume per unit area of the first pores is greater than or equal to the breathable volume per unit area of the second pores.

Preferably, the porosity of the first pores is different from the porosity of the second pores.

In one embodiment of the present invention, the PET fiber fixation layer and the thermoplastic elastomer (TPE) microporous layer can be bonded together by hot press fusion or high frequency bonding to form the breathing film. Further, the TPU material of the breathing film can be bonded to the rim around the top opening of the PCTG cap.

Further, the essential oil substance can be a pure concentrated hydrophobic liquid containing volatile aroma compounds from plants, a concentrated hydrophobic liquid containing volatile synthetic aroma chemical compounds added with a diffusion agent or diluted with a fluid.

In general, the invention achieves the effects as follows: The breathing film allows the aromatic molecular vapor generated by the essential oil substance to flow to the environment outside the bottle essential oil diffuser, and can prohibit the essential oil substance from flowing out of the bottle essential oil diffuser. When using the heater to heat the bottle essential oil diffuser, the breathing film does not shrink and will not cause wrinkles. The aromatic molecular vapor generated by the essential oil substance can effectively flow through the breathing film to the outside of the bottle essential oil diffuser. When compared with conventional aromatic diffuser designs, the bottle essential oil diffuser of the invention has better ventilation effect for enabling the generated aromatic molecular vapor to effectively flow to the outside air. Even if the heater or the bottle essential oil diffuser falls down accidentally, the breathing film can effectively prohibit the fluid essential oil substance from flowing out of the top opening of the cap, avoiding contamination.

### BRIEF DESCRIPTION OF THE DRAWINGS

FIG. 1 is an oblique top elevational view of a bottle essential oil diffuser in accordance with the present invention.

FIG. 2 is an exploded view of the bottle essential oil diffuser in accordance with the present invention.

FIG. 3 is a cross sectional view of the bottle essential oil diffuser in accordance with the present invention.

FIG. 4 illustrates the outer appearance of the cap and the breathing film.

FIG. 5 corresponds to FIG. 4, illustrates the breathing film assembled.

FIG. 6 is a sectional view of the cap.

FIG. 7 is a schematic applied view of the present invention, illustrating a hand tool inserted into one notch of the cap for opening the cap.

FIG. 8 is an elevational view illustrating the cap separated from the bottle.

FIG. 9 is an elevational view of an electrical plug type heater for use with the bottle essential oil diffuser in accordance with the present invention.

FIG. 10 illustrates the bottle of the bottle essential oil diffuser set in the electrical plug type heater.

FIG. 11 is an elevational view of a table-top heater for use with the bottle essential oil diffuser in accordance with the present invention.

FIG. 12 illustrates the bottle essential oil diffuser set in the table-top heater.

FIG. 13 is an oblique top elevational view of a perforated protective cover in accordance with the present invention.

FIG. 14 illustrates an alternate form of the perforated protective cover.

### DETAILED DESCRIPTION OF THE PREFERRED EMBODIMENT

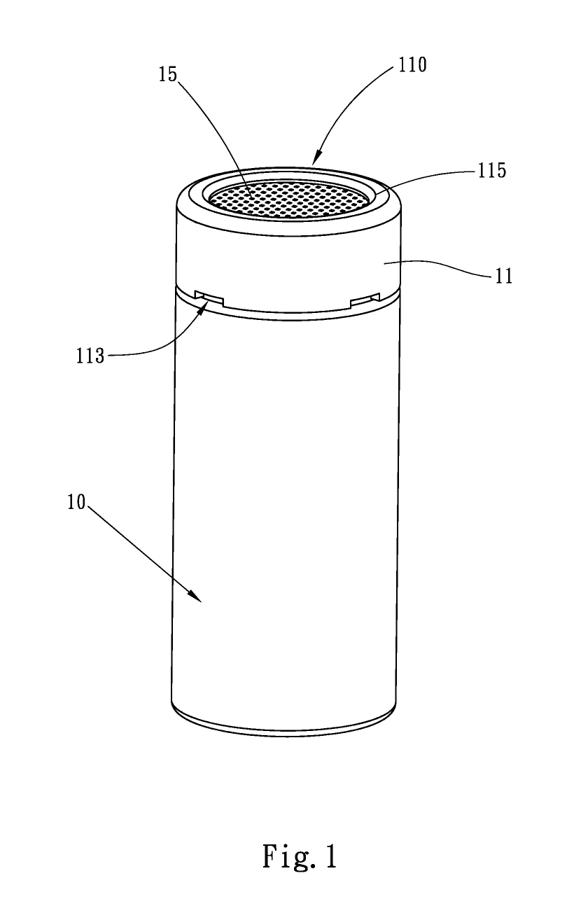

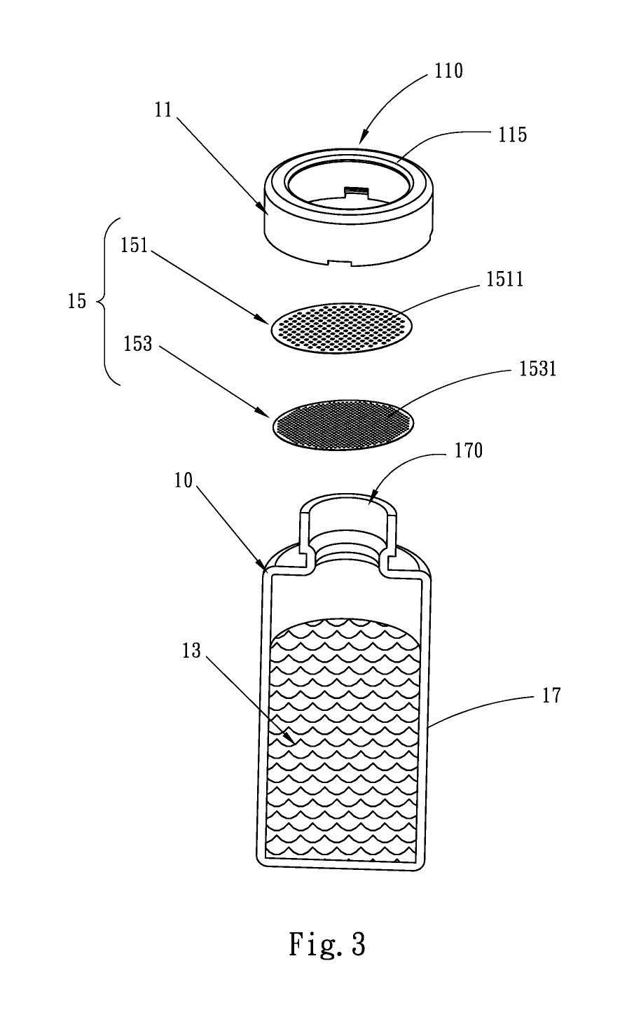

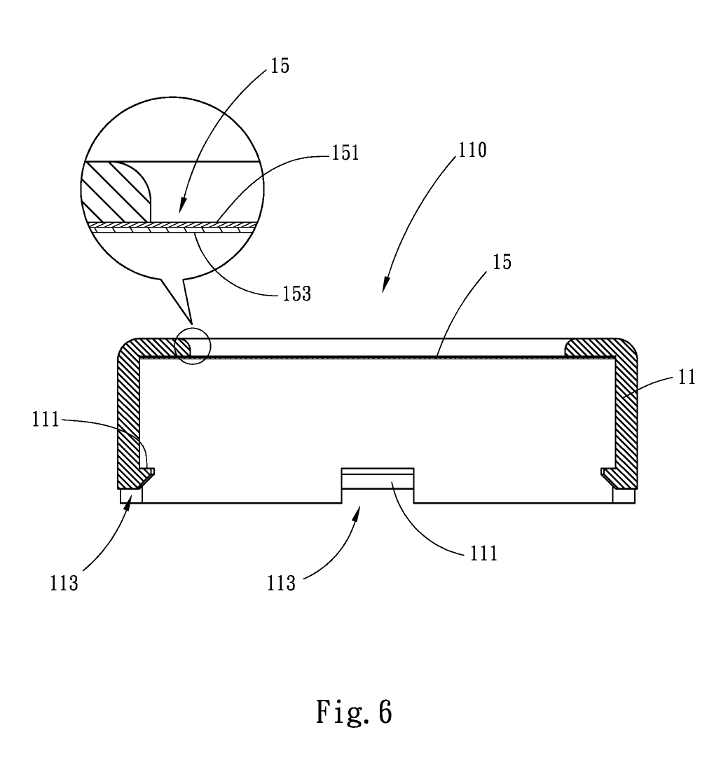

Referring to FIGS. 1-6, a bottle essential oil diffuser with a breathing film incorporated cap in accordance with the present invention is shown. The bottle essential oil diffuser 10 comprises a cap 11, an essential oil substance 13, a breathing film 15, and a bottle 17. The cap 11 comprises a top opening 110. The bottle 17 comprises a bottle mouth 170. The essential oil substance 13 is put in the bottle 17. The breathing film 15 is bonded to the top opening 110 of the cap 11. The cap 11 is capped on the bottle mouth 170 of the bottle 17 and covered over the essential oil substance 13. The breathing film 15 has a breathable function.

The breathing film 15 comprises a fiber fixation layer 151 and a thermoplastic elastomer (TPE) microporous layer 153. The fiber fixation layer 151 comprises a plurality of first pores 1511. The thermoplastic elastomer (TPE) microporous layer 153 comprises a plurality of second pores 1531. The fiber fixation layer 151 is bonded to the thermoplastic elastomer (TPE) microporous layer 153 to form the breathing film 15. The thermoplastic elastomer (TPE) microporous layer 153 has a waterproof and moisture-permeable function. The fiber fixation layer 151 is heat and deformation resistant. The breathing film 15 allows the aromatic molecules of the essential oil substance 13 to flow to the outside of the bottle essential oil diffuser and prohibits the essential oil substance from flowing out of the bottle essential oil diffuser during application.

In one embodiment of the present invention, the bottle essential oil diffuser 10 is placed in a heater 1 and heated to generate an aromatic vapor. The fiber fixation layer 151 does not shrink or deform in the heating process, and can hold the thermoplastic elastomer (TPE) microporous layer 153 in shape. The surface of the breathing film 15 does not shrink or deform to cause wrinkles. In the heating process, the essential oil substance 13 generates an aromatic molecular vapor of a certain concentration that can pass through the micro open spaces in the thermoplastic elastomer (TPE) microporous layer 153 and the first pores 1511 of the fiber fixation layer 151 toward the low concentration air outside the bottle essential oil diffuser 10. The breathing film allows the aromatic molecular vapor generated by the essential oil substance 13 to effectively flow to the environment outside the bottle essential oil diffuser 10. Even if the bottle essential oil diffuser 10 falls down during delivery or heating, the fluid of the essential oil substance 13 in the bottle essential oil diffuser 10 is prohibited from flowing through the thermoplastic elastomer (TPE) microporous layer 153 of the breathing film 15 to the outside. Therefore, the bottle essential oil diffuser with a breathing film incorporated cap in accordance with the present invention is more saver in use when comparted to prior art designs.

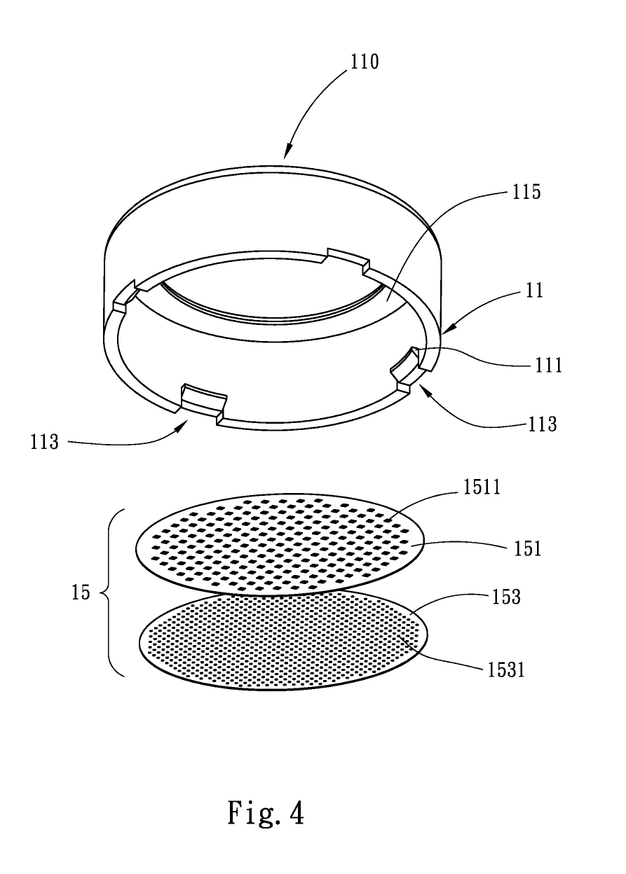

Referring to FIGS. 4-6, the fiber fixation layer 151 defines therein multiple first pores 1511. The microporous layer 153 defines therein multiple second pores 1531. The fiber fixation layer 151 and the microporous layer 153 are bonded together to form the breathing film 15 with the breathable and waterproof function. The microporous layer 153 is preferably made from thermoplastic elastomer (TPE) compound. The fiber fixation layer 151 is preferably made from polyester. The polyester fiber fixation layer 151 and the thermoplastic elastomer (TPE) microporous layer 153 are bonded together to form the breathing film 15 that is breathable. The breathable aperture of the first pores 1511 is greater than the breathable aperture of the second pores 1531, facilitating outward transmittance of air. The breathing film 15 is bonded to the cap 11 over the top opening 110. Through the second pores 1531 of the thermoplastic elastomer (TPE) microporous layer 153, the cap 11 provides waterproof and moisture transmissive functions. In application, the bottle essential oil diffuser 10 prohibits the contained essential oil substance 13 from flowing toward the outside, ensuring a high level of safety.

The thermoplastic elastomer (TPE) microporous layer 153 and the fiber fixation layer 151 can be bonded together using hot-press fusion or high-frequency sealing technology. Since the first pores 1511 of the fiber fixation layer 151 are relatively lager than the second pores 1531 of the thermoplastic elastomer (TPE) microporous layer 153, the heated aromatic substance vapor molecules can go through second pores 1531 of the thermoplastic elastomer (TPE) microporous layer 153 toward the first pores 1511 of the fiber fixation layer 151.

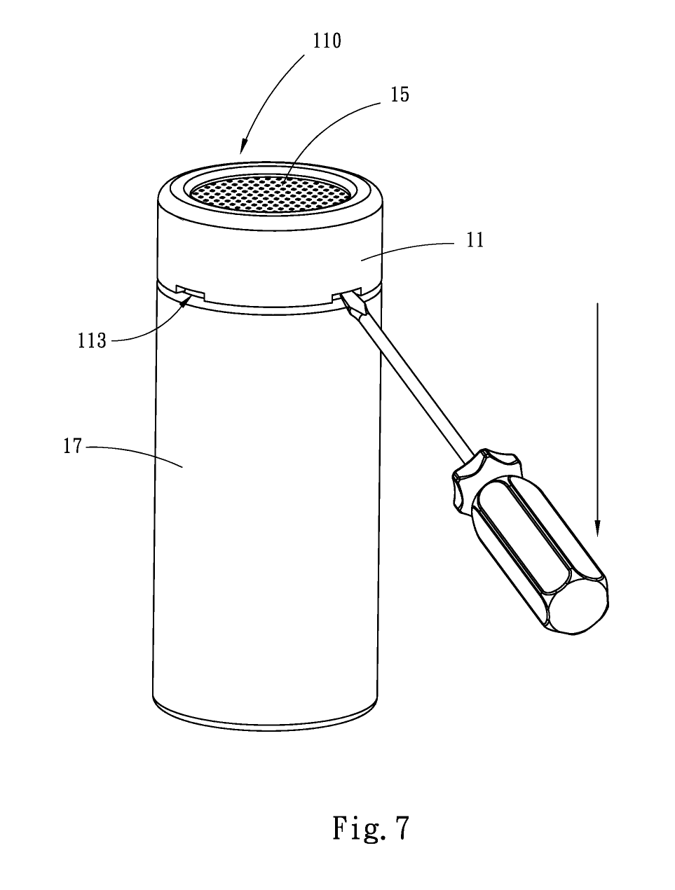

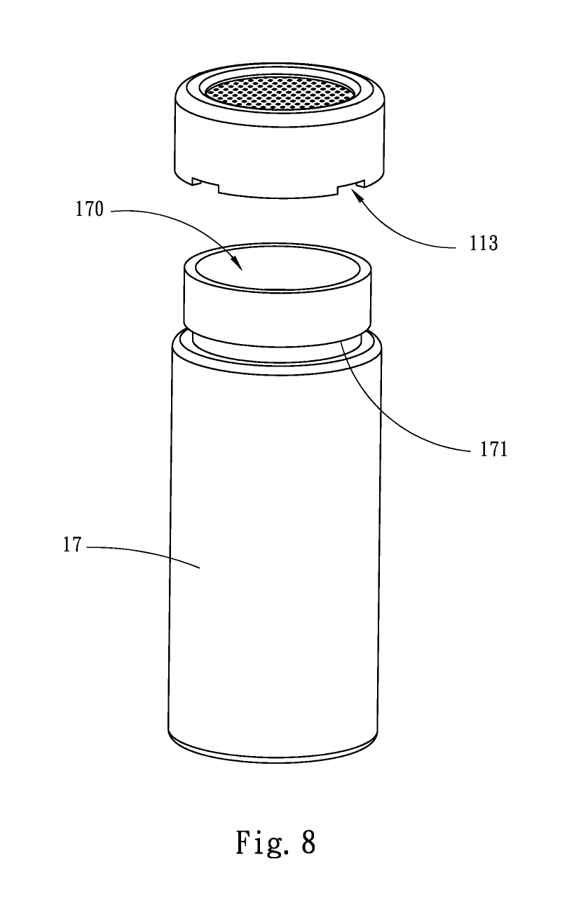

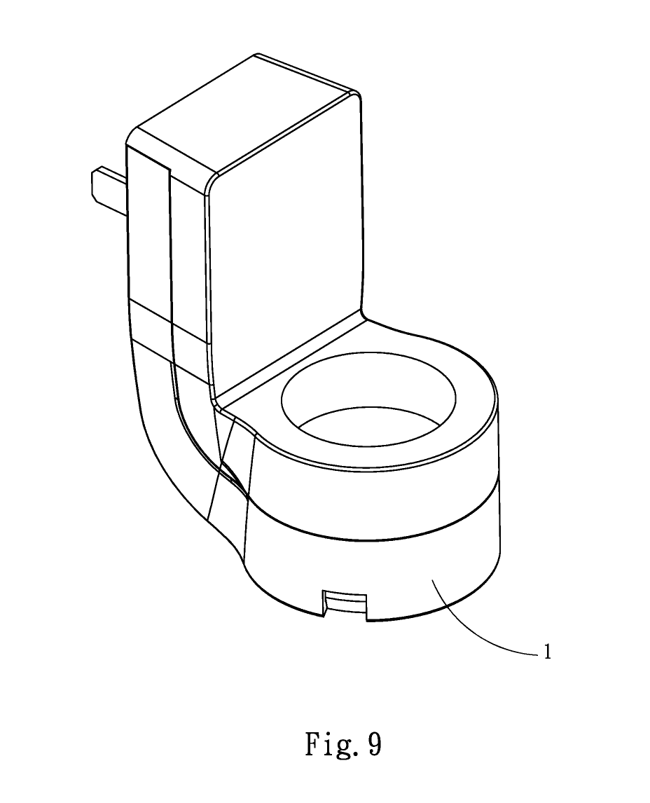

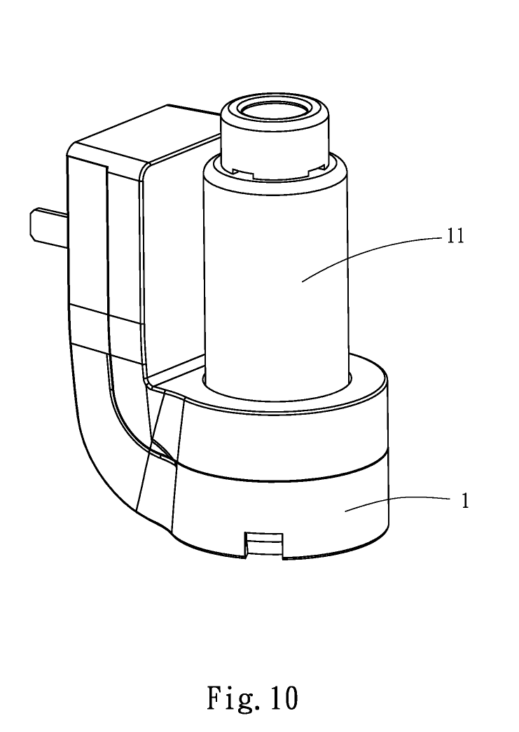

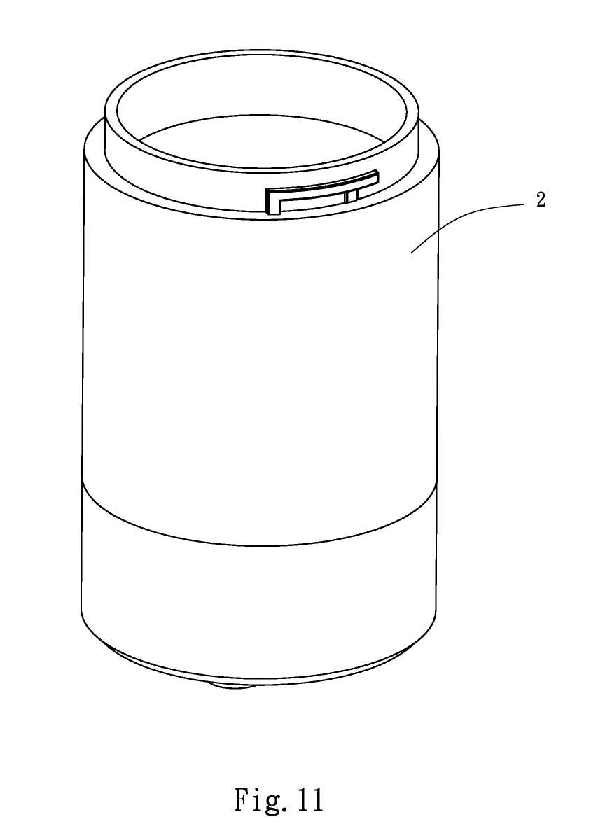

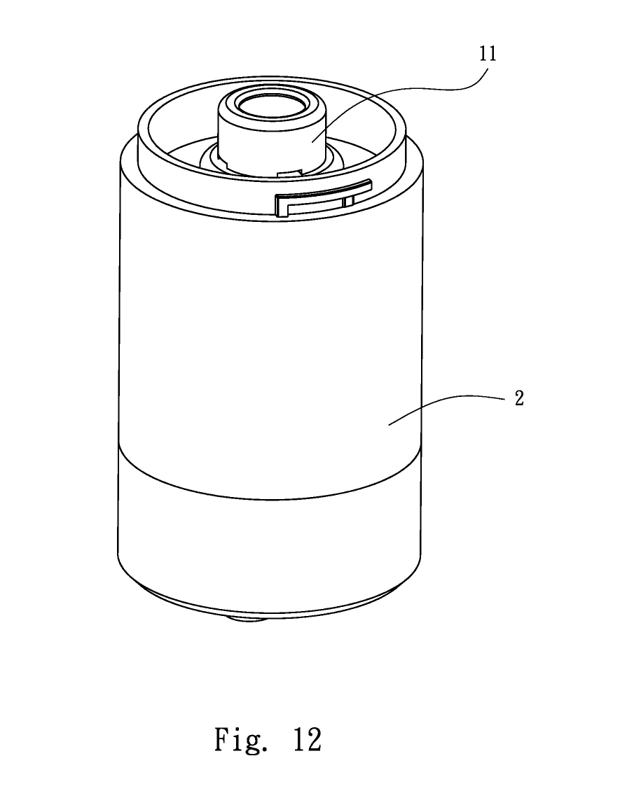

Referring to FIG. 1 and FIGS. 7-10, the bottle essential oil diffuser with a breathing film incorporated cap can be an electrical plug type heater 1 (see FIG. 9 and FIG. 10) or a table-top heater 2 (see FIG. 11 and FIG. 12). When the bottle essential oil diffuser 10 is put in the heater 1 and heated, the essential oil substance 13 is heated to generate an aromatic molecular vapor. In the heating process, the fiber fixation layer 151 does not shrink or deform and can hold the thermoplastic elastomer (TPE) microporous layer 153 constantly in shape, and thus, the surface of the breathing film 15 does not deform and will not cause wrinkles. In the heating process, the aromatic molecular vapor generated by the essential oil substance 13 flows smoothly through the second pores 1531 of the thermoplastic elastomer (TPE) microporous layer 153 and the first pores 1511 of the fiber fixation layer 151 toward the low concentration air outside the bottle essential oil diffuser 10, however, the fluid of the essential oil substance 13 is prohibited from flowing through the second pores 1531 to the outside of the bottle essential oil diffuser 10. Thus, even if the bottle essential oil diffuser 10 falls down, the essential oil substance 13 will not leak out, ensuring a high level of safety.

The essential oil substance 13 can be a pure concentrated hydrophobic liquid containing volatile aroma compounds from plants, a concentrated hydrophobic liquid containing volatile synthetic aroma chemical compounds added with a diffusion agent or diluted with a fluid.

In certain embodiments of the present invention, the cap 11 or bottle 17 is made from polymers. In one embodiment of the present invention, the cap 11 or bottle 17 is made from a plastic material selected from the group of polyester plastic, poly-cyclohexylenedimethulene terephthalate glyco (PCTG), polyethylene terephthalate (PET), poly-thiourethane (PTU) and polypropylene (PP). The plastic cap 11 or bottle 17 will not melt or deform in the above-mentioned heating temperature, and can transfer the thermal energy generated by the heater 1 or heater 2 to the essential oil substance 13. In one preferred embodiment of the present invention, the cap 11 or bottle 17 is made from PCTG and can transfer heat source from a heat sink 10 to the essential oil substance 13, causing the essential oil substance 13 to generate an aromatic vapor. Unlike the existing essential oil bowls, glass containers and other fragile products, the plastic cap 11 or bottle 17 is ductile, tough and not easy to break, will not damage when heated by the heater, and can transfer heat source to the essential oil substance 13. Further, the top opening 110 of the plastic cap 11 facilitates bonding of the breathing film 15, for example, the thermoplastic elastomer (TPE) microporous layer 153 of the breathing film 15 can be fused with the top opening 110 of the plastic cap 11, or bonded with the top opening 110 of the plastic cap 11 using an adhesive.

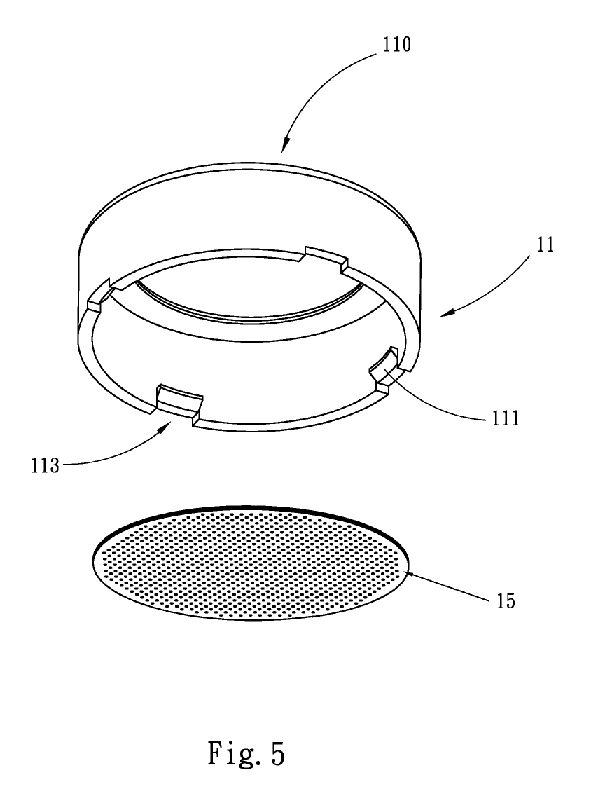

Referring to FIGS. 5-8, in one embodiment of the present invention, the cap 11 comprises a plurality of hooks 111; the bottle 17 comprises an endless hook groove 171 extended around a bottom side of the periphery of the bottle mouth 170. When capping the cap 11 on the bottle mouth 170 of the bottle 17, the hooks 111 are hooked in the hook groove 171 to secure the cap 11 firmly to the top side of the bottle mouth 170 of the bottle 17.

Referring to FIGS. 5-8, in one embodiment of the present invention, the cap 11 further comprises a plurality of notches 113 spaced around a bottom edge thereof and respectively disposed at a bottom side relative to the respective hooks 11. The user can use a hand tool, such as a flat tip screwdriver to open the cap 11 from the bottle 17 by means of inserting the flat tip of the flat tip screwdriver into one notch 113 and then bias the flat tip screwdriver to disengage the respective hook 111 from the hook groove 171. After removal of the cap 11 from the bottle 17, the user can refill essential oil or water in the essential oil diffuser 10.

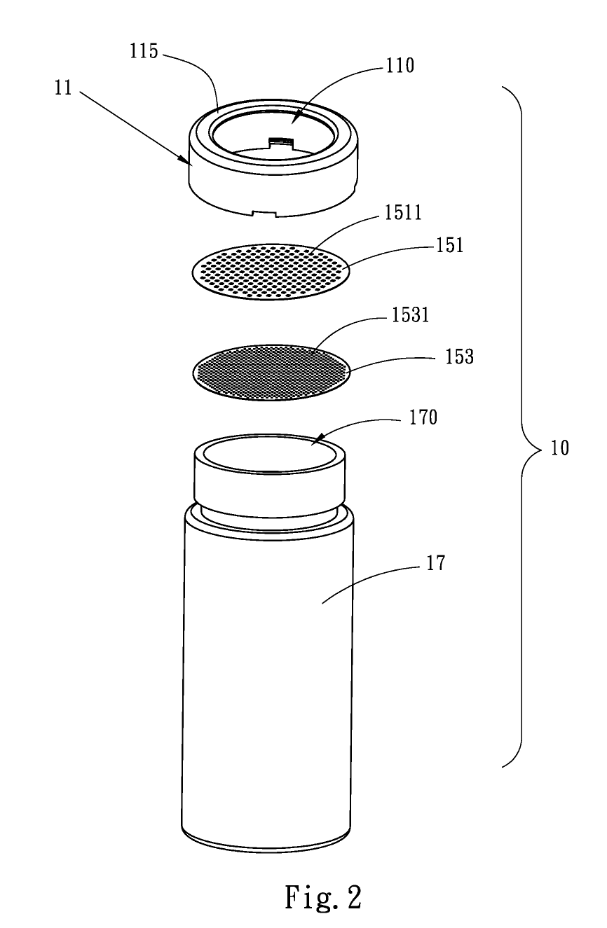

Referring to FIGS. 2, 6 and 7, the cap 11 further comprises an inner rim 115 extending around the inner perimeter of the top opening 110. In this embodiment, the inner rim 115 exhibits an annular shape. The breathing film 15 is bonded to the inner rim 115 of the cap 11 over the top opening 110.

Referring to FIGS. 4, 5 and 6, in one embodiment of the present invention, the cap 11 is a plastic cap 11; the fiber fixation layer 151 is made out of PET; the thermoplastic elastomer (TPE) microporous layer 153 is made out of thermoplastic polyurethane (TPU); the PET fiber fixation layer 151 and the thermoplastic elastomer (TPE) microporous layer 153 are bonded together by hot press bonding or high-frequency sealing without affecting the breathing film 15; the TPU microporous layer 153 of the breathing film 15 is peripherally bonded to the top opening 110 of the plastic (such as PCTG) cap 11 by hot press bonding or high-frequency sealing; the TPU microporous layer 153 is peripherally bonded to the rim 115 of the PCTG cap 11 over the top opening 110, and covered over the essential oil 13 to seal the essential oil 13 in the bottle essential oil diffuser 10.

Referring to FIGS. 4, 5 and 6, in one embodiment of the present invention, the plastic cap 11 is made out of PCTG; the fiber fixation layer 151 of the breathing film 15 is made out of PET; the microporous layer 153 of the breathing film 15 is made out of TPU; subject to the fusible nature between PTU and PCTG, the TPU layer of the breathing film 15 is peripherally bonded to the rim 115 of the PCTG cap 11 around top opening 110 by an automatic machine, improving the bonding quality and production rate and saving the cost. Further, an adhesive can be used to bond the breathing film 15 and the cap 11 together.

Referring to FIGS. 4, 5 and 6, the fiber fixation layer 151 and microporous layer 153 of the cap 11 can be swapped upside down. In this case, the top side of the fiber fixation layer 151 is bonded to the microporous layer 153 to create the breathing film 15; the fiber fixation layer 151 is peripherally bonded to the cap 11 over the top opening 110; when the essential oil 13 (such as aromatic essential oil) is being heated, the emitted aromatic vapor particles go through the first pores 1511 of the fiber fixation layer 151 and then the second pores 1531 of the microporous layer 153 toward the outside open air; the effect produced by the breathing film 15 is same is same as described above.

Referring to FIGS. 4, 5 and 6, in one embodiment of the present invention, the breathable aperture of the first pores 1511 of the fiber fixation layer 151 is greater than or equal to the breathable aperture of the second pores 1531 of the microporous layer 153, facilitating passing of the generated aromatic vapor molecules through the breathing film 15, i.e., the ventilatory capacity per unit area of the first pores 1511 of the fiber fixation layer 151 is greater than or equal to the ventilatory capacity per unit area of the second pores 1531 of the microporous layer 153, facilitating passing of released aromatic vapor molecules through the breathing film 15.

Referring to FIGS. 4, 5 and 6, in one embodiment of the present invention, the porosity of the first pores 1511 of the fiber fixation layer 151 and the porosity of the second pores 1531 of the microporous layer 153 are unequal.

The concentration of aromatic molecular vapor generated by the essential oil substance 13 that is heated by the heater 1 or heater 2 is higher than the concentration of air outside the breathing film 15 of the bottle essential oil diffuser 10, the high concentration of aromatic molecular vapor will fly through the second pores 1531 of the TPU thermoplastic elastomer (TPE) microporous layer 153 and the first pores 1511 of the PET fiber fixation layer 151 to the low concentration of air outside the bottle essential oil diffuser 10.

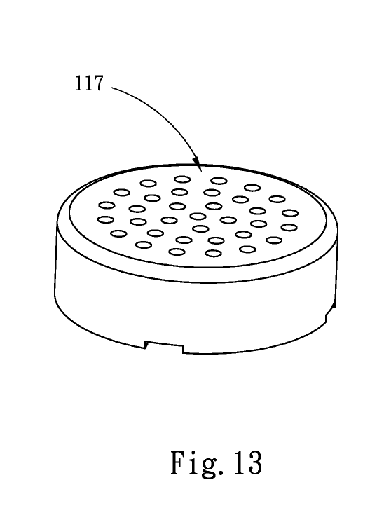

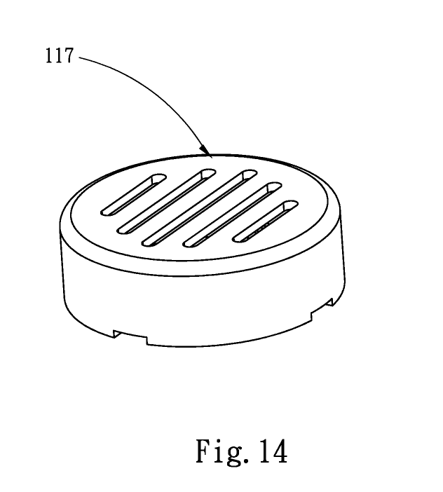

Referring to FIG. 13 and FIG. 14, in one embodiment of the present invention, the bottle essential oil diffuser 10 further comprises a perforated protective cover 117 capped on the top opening 110 of the cap 11. The perforated protective cover 117 can be configured to provide circular through holes (see FIG. 13) or elongated through holes (see FIG. 13). The perforated protective cover 117 protects the breathing film 15 against damage, allowing the aromatic molecular vapor generated by the essential oil substance 13 to pass therethrough to the outside open air.

Although particular embodiments of the invention have been described in detail for purpose of illustration, various modifications and enhancements may be made without departing from the spirit and scope of the invention. Accordingly, the invention is not to be limited except as by the appended claims.

What the invention claimed is:

1. A bottle essential oil diffuser, comprising a bottle having a bottle mouth, an essential oil substance contained in said bottle, a cap capped on said bottle mouth of said bottle and defining therein a top opening and a breathing film bonded to said cap over said top opening, said breathing film comprising a fiber fixation layer and a microporous layer bonded together, said fiber fixation layer comprising a plurality of first pores, said microporous layer comprising a plurality of second pores.

2. The bottle essential oil diffuser as claimed in claim 1, further comprising a heater adapted for holding and heating said bottle, causing said essential oil substance to generate an aromatic vapor.

3. The bottle essential oil diffuser as claimed in claim 1, wherein said microporous layer is made from thermoplastic elastomer (TPE); said fiber fixation layer is made from polyester.

4. The bottle essential oil diffuser as claimed in claim 3, wherein said polyester is selected from the group consisting of Tetoron and polyethylene terephthalate (PET); said thermoplastic elastomer (TPE) is thermoplastic polyurethane (TPU).

5. The bottle essential oil diffuser as claimed in claim 4, wherein said cap is made from a plastic material selected from the group consisting of polyester plastic, poly-cyclohexylenedimethulene terephthalate glyco (PCTG), polyethylene terephthalate (PET), poly-thiourethane (PTU) and polypropylene (PP); said microporous layer of said breathing film is bonded to said top opening of said cap.

6. The bottle essential oil diffuser as claimed in claim 1, wherein the breathable aperture of said first pores is greater than the breathable aperture of said second pores.

7. The bottle essential oil diffuser as claimed in claim 1, wherein the porosity of said first pores is different from the porosity of said second pores.

8. The bottle essential oil diffuser as claimed in claim 1, wherein said bottle comprises a hook groove extended around a bottom side of the periphery of said bottle mouth; said cap further comprises a plurality of hooks hooked in said hook groove of said bottle, and a plurality of notches spaced around a bottom edge thereof and respectively disposed at a bottom side relative to the respective said hooks.

9. The bottle essential oil diffuser as claimed in claim 8, wherein said cap further comprises a rim extended around an inner perimeter of said top opening; said breathing film is bonded to said rim of said cap over said top opening.

10. The bottle essential oil diffuser as claimed in claim 1, further comprising a perforated protective cover capped on said cap over said top opening.
